(19) United States
(12) Patent Application Publication
(10) Pub. No.: US 2006/0115836 A1
(43) Pub. Date: Jun. 1, 2006

(54) Utilization of FANCC, DAD1, GRIM19 and HADHII genes for the ultimate treatment of psoriasis

(75) Inventors: Valerie Trinquet, Antibes (FR); Patricia Rossio, Plas Cassier (FR); Paul Fogel, Paris (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA RESEARCH & DEVELOPMENT, S.N.C., VALBONNE SOPHIA ANTIPOLIS (FR)

(21) Appl. No.: 11/249,305

(22) Filed: Oct. 14, 2005

Related U.S. Application Data
(63) Continuation of application No. PCT/FR04/00934, filed on Apr. 15, 2004.

(30) Foreign Application Priority Data
Apr. 16, 2003 (FR) 03/04773

(51) Int. Cl. C12Q 1/68; C07H 21/04; C12P 21/06; C12N 9/64

(52) U.S. Cl. 435/6; 435/69.1; 435/226; 435/320.1; 435/325; 530/388.26; 536/23.2

(57) ABSTRACT

Polynucleotide probes relating to the FANCC, DAD1, GRIM19 and HADHII genes are useful for characterizing biological samples and for identifying compounds suited for the treatment of psoriasis.

### CROSS-REFERENCE TO PRIORITY/PCT APPLICATIONS

This application claims priority under 35 U.S.C. § 119 of FR 03/04773, filed Apr. 16, 2003, and is a continuation of PCT/FR 2004/000934, filed Apr. 15, 2004 and designating the United States (published in the French language on Nov. 4, 2004 as WO 2004/094661 A2; the title and Abstract were also published in English), each hereby expressly incorporated by reference and each assigned to the assignee hereof.

### BACKGROUND OF THE INVENTION

1. Technical Field of the Invention

The present invention relates to the use of probes relating to the FANCC, DAD1, GRIM19 and HADHII genes for characterizing a biological sample.

This invention also relates to the use of these probes for identifying a compound for use in the treatment of psoriasis, to a method of identification and also to a novel research tool.

2. Description of Background and/or Related and/or Prior Art

Psoriasis is a common dermatological pathology which affects 3 to 5% of the European population. Psoriasis is commonly associated with other pathologies, such as hypertension, arthritis, or AIDS (Faber E M, Natl, In Roenigk H H, Maibach H I (Eds.): Psoriasis Third Edition, Revised and Expanded (1998), Chapter 9:107-157; Duvic M., J. Invest. Dermatol., (1990 November); 95(5):38S-40S).

Based on the age of appearance of the symptoms and on the association with certain genes of the type I major hisocompatibility complex (MHC I), psoriasis has been classified into two subtypes referred to as I and II (Henseler T, Christophers E, In Roenigk H H, Maibach H I (Eds.): Psoriasis Third Edition, Revised and Expanded (1998), chapter 10:159-166). Studies have also shown that 30 to 50% of patients suffering from psoriasis have a 1 st or 2nd degree family history in one of the parents. These observations have made it possible to suggest that psoriasis is a genetic disease with an autoimmune component (Bos, Br. J. Dermtol., (1988) 188:141: Baadsgaard et al., J. Invest. Dermatol., (1990) 95:328).

The main physiopathological components of psoriasis include hyperproliferation associated with abnormal differentiation of epidermal keratinocytes, inflammation characterized by an inflammatory infiltrate (mainly of activated helper T lymphocytes) in the dermis and the epidermis and by the release of pro-inflammatory cytokines, and, finally, abnormal dermal vascularization (Krueger J G, J. Am. Acad. Dermatol.,(2002 January);46(1):1-23; quiz 23-6).

The involvement of the inflammatory process in the physiopathology of psoriasis is supported by the mechanism of action of certain agents that have a beneficial therapeutic effect on the disease. For example, cyclosporin used in the treatment of severe forms of psoriasis exerts an inhibitory effect on the synthesis of interleukin-2, a cytokine that promotes T lymphocyte proliferation and the secretion of inflammatory cytokines by these cells. Consequently, inflammation modulators make it possible to limit the inflammatory component that partly characterizes psoriasis.

Clinically, psoriasis is defined by hyperplasia of the epidermis, and by desquamation, erythema, and skin inflammation.

The methods currently available for treatment include topical therapy, phototherapy, photochemotherapy and systemic therapy, reserved for the most severe forms of the disease. However, these therapies offer the patients only periods of remission, and the pathology generally flares up again after the treatment has been stopped.

Glucocorticoids administered topically are most generally prescribed in the initial treatment of psoriasis, and exert beneficial anti-inflammatory, antimitotic and antipruritic effects.

Crude coal tar is a complex mixture of thousands of hydrocarbon compounds. Its therapeutic effect is related to the inhibition of enzymes and to antimitotic properties. Although effective, this treatment is, because of its odor, unpleasant for the patient to use and can induce skin cancers.

Phototherapy and photochemotherapy, based on the administration of methoxsalen, a photosensitizing active principle, are only temporarily effective. The treatment must be repeated and offers palliative, but not curative, therapy. In addition, these treatments have long-term side effects, in particular immunological impairments and an increased risk of carcinogenesis.

Methotrexate is the systemic cytotoxic agent most generally administered for severe forms of psoriasis that extend over a substantial surface of the body. There are many contraindications for this treatment and interruption of the latter can be accompanied by worsening of the symptoms. Hydroxyurea, etretinate, cyclosporin and AZT are also systemic medicaments used to combat psoriasis, but they all have serious side effects.

To date, no treatment allows long-lasting remission of this pathology without engendering considerable side effects, because psoriasis is a complex pathology of multifactor origin. Understanding the physiopathological mechanism that leads to the psoriatic phenotype therefore remains an important challenge in dermatological medical treatment. This is because many aspects of the disease remain to be elucidated, for instance the heterogeneous response to certain treatments of patients exhibiting similar clinical symptoms.

Apoptosis is a phenomenon of cell death (described, inter alia, by KERR J. F. R. et al., J. Cancer, (1972), 265, 239) that is a highly selective form of cell suicide. It is characterized by readily observable morphological and biochemical phenomena. Thus, chromatin condensation possibly associated with endonuclease activity, the formation of apoptotic bodies, and fragmentation of deoxyribonucleic acid (DNA), due to the activation of endonucleases, into DNA fragments of 180-200 base pairs, giving a readily recognizable profile by agarose gel electrophoresis, are observed.

Apoptosis is involved in tissue development, differentiation and renewal.

In view of the knowledge of the physiological process of cellular apoptosis, nothing allows those skilled in the art to deduce any involvement thereof in the physiological disregulation that leads to the psoriatic phenotype.

The analysis of gene expression in the skin of psoriatic patients constitutes an approach for obtaining a better understanding of the physiopathological mechanism of this disease (Bowcock A M, Shannon W, Du F, et al., Hum. Mol. Genet., (2001); 10(17):1793-805; Oestreicher J L, Walters I B, Kikuchi T, et al., Pharmacogenomics J., (2001); 1(4):272-87).

cDNA filter (array) technology (Nguyen C, Rocha D, Granjeaud S, et al., Genomics, (1995)) is one of the technologies for simultaneously and rapidly analyzing the expression of several hundred mRNAs from a skin biopsy taken form the patient, as opposed to other technologies that are more laborious to implement, such as SAGE (Serial Analysis of Gene Expression) (Velculescu V E, Zhang L, Vogelstein B, Kinzler K W, Science, (1995 October) 20;270(5235):484-7), or Differential Display (Liang P, Pardee A B, Science, (1992); 257:967-971; Zhang L, et al., Science, (1997); 276:1268-1272).

### SUMMARY OF THE INVENTION

Novel marker genes for the ultimate treatment of psoriasis have now been determined.

Thus, the present invention features probes relating to the FANCC, DAD1, GRIM19 and HADHII genes for characterizing a biological sample, and also a novel tool for characterizing a compound for use in the treatment of psoriasis, and also a novel research tool.

In particular, this invention relates to the use of at least one polynucleotide probe relating, respectively, to the FANCC, DAD1, GRIM19 and HADHII gene, that has a sequence of 10 to 500 base pairs and is at least 80% complementary with a target sequence corresponding to all or part, respectively, of the FANCC gene (accession no. NM 000136.1), DAD1 gene (accession no. NM 001344.1), GRIM19 gene (accession no. NM 015965.3) or HADHII gene (accession no. NM 004493.1), as a research tool for psoriasis.

The use of at least one polynucleotide probe according to the invention makes it possible in particular to characterize a biological sample by measuring the level of expression of one or more genes selected from FANCC, DAD1, GRIM19 and HADHII.

### DETAILED DESCRIPTION OF BEST MODE AND SPECIFIC/PREFERRED EMBODIMENTS OF THE INVENTION

According to the present invention, a biological sample is defined as any specimen or sample from a living organism, preferably a mammal, in particular a human organism, in sufficient amount to be characterized. As a biological sample, mention may be made, by way of non-limiting example, of a skin sample, a scalp sample, a mucosal sample or a cell sample.

According to the present invention, the characterization of a biological sample entails measuring the level of expression of a gene of interest, i.e., more precisely the amount of messenger RNA or of protein corresponding to this gene which is present in the biological sample.

The term “polynucleotide probe” means a single-stranded or double-stranded oligonucleotide or polynucleotide of RNA or DNA, the sequence of which is complementary to a target sequence corresponding to part or to all of said gene and which can range from 10 to conventionally 500 base pairs, and the sequence of which is at least 80%, and preferably at least 90%, complementary with the target sequence (percentage of paired nucleic acid bases between two sequences, see the calculation methods proposed by Altschul et al., J. Molec. Biol., (1990), 215:403).

The polynucleotide probes may also have a modified backbone that confers, for example, better stability thereon.

For example, the phosphodiester bonds of natural RNA strands can be modified to include at least one nitrogen or sulfur atom.

Moreover, the polynucleotide probes may be modified such that their phosphodiester bonds are replaced with peptide-type sequences (peptide nucleic acids).

In addition, the polynucleotide probes according to the invention can comprise bases other than the 4 conventional bases.

The polynucleotide probes according to the invention can be synthesized according to many methods of synthesis in vivo or in vitro, manually or automatically.

The in vitro methods of synthesis may be chemical or enzymatic. For example, a single-stranded cRNA probe can be obtained by in vivo transcription of a model of a DNA sequence of interest acting as a template, using an RNA polymerase (by way of example, T3, T7 or SP5 RNA polymerase).

A single-stranded cDNA probe can be generated by reverse transcription of an mRNA of interest in the presence of a reverse transcriptase (for example, M-MLV (Moloney Murine Leukaemia Virus) reverse transcriptase isolated from Escherichia coli).

Many methods of in vivo synthesis of double-stranded RNA are described in the literature; they can be carried out in various bacterial cell types or cell types of higher organisms. Reference may also be made to the methods of synthesis described in WO 01/36646 and WO 01/75164. These double-stranded RNAs can then be denatured so as to serve as a single-stranded polynucleotide.

As regards its environment, the polynucleotide probe may be or may be part of a plasmid, a viral genome or another type of vector. It may, in other cases, be part of the genome of a cell, or else of a cell that has been genetically modified so as to contain this probe in its genome. It may also be an isolated molecule.

As regards the regions surrounding this probe, it is preferably under the control of regulatory sequences. If the probe is inserted into a vector, said vector preferably comprises all the sequences required for the transcription and, optionally, the translation of the probe. This probe can also be surrounded by flanking regions that allow a step consisting of homologous recombination with another polynucleotide fragment, optionally resulting in the insertion of a probe of the invention into the genomic DNA of a target cell.

The present invention also features the use of at least one polypeptide probe relating to the expression product of a gene selected from among FANCC, DAD1, GRIM19 and HADHII, as a research tool for psoriasis.

The use of at least one polypeptide probe according to the invention makes it possible in particular to characterize a biological sample by measuring the level of expression of the FANCC and/or DAD1 and/or GRIM19 and/or HADHII gene.

The expression “polypeptide probe relating to the expression product of the FANCC, DAD1, GRIM19 or HADHII gene” means a monoclonal or polyclonal antibody specifically directed against the wild-type protein derived from the expression, respectively, of the FANCC, DAD1, GRIM19 or HADHII gene, or against a biologically active fragment of said protein, or else against a homologous, modified or variant polypeptide of the wild-type protein. Said antibody can be obtained according to the methods known to those skilled in the art (Catty D (Ed): Antibodies, A Practical Approach, Volume 1 (1989), chapters 2, 3 and 4; or else Harlow E. and Lane D., Antibodies, A laboratory Manual, (1988), chapters 6 and 7) using said wild-type protein or said homologous polypeptide.

This may, for example, involve a monoclonal or polyclonal antibody, or their biologically active fragment capable of recognizing the protein encoded by the gene of interest. These antibodies may be labeled, for example immunoconjugated to enzymes or labeled with fluorescent compounds or biotin, or else radio labeled.

The term “homologous polypeptides” means polypeptides whose amino acid sequences exhibit a minimum of 80%, and preferably 90%, of amino acids in common with the wild-type protein encoded by the gene of interest.

The term “variant polypeptide” is intended to denote a mutated polypeptide or a polypeptide corresponding to a polymorphism that may exist, in particular in human beings, and that may exhibit a truncation, a substitution, a deletion and/or an addition of at least one amino acid compared with the sequence of the normal wild-type polypeptide.

The term “modified polypeptide” is intended to denote a polypeptide obtained by genetic recombination or by chemical synthesis, exhibiting a modification compared with the normal wild-type sequence. These modifications may in particular relate to the pre-, pro- or mature domains of the polypeptides, or to the amino acids responsible for a spectrum specificity or for effectiveness of the activity, or responsible for the structural conformation, for the charge or the hydrophobicity, and for the capacity for multimerization and for membrane insertion of the polypeptides. It will thus be possible to create polypeptides having equivalent activity that is stricter or broader. The modifications may also relate to the sequences involved in the maturation, the transport and the targeting of the polypeptides.

The expression “biologically active fragment of a polypeptide” is intended to denote a polypeptide fragment that has conserved at least one activity of the polypeptide from which it is derived.

A comparative study of gene expression in psoriatic skin compared with normal skin has enabled the applicant to identify four genes, called FANCC (Fanconi anaemia, complementation group C), Genbank reference (NCBI): NM_{—}000136.1, DAD1 (defender against cell death 1) Genbank reference (NCBI): NM_{—}001344.1, GRIM19 (gene associated with retinoid-interferon-induced mortality 19) Genbank reference (NCBI): NM_{—}015965.3 and HADHII (hydroxyacyl-coenzyme A dehydrogenase, type II) Genbank reference (NCBI): NM_{—}004493.1, the transcriptional activity of which is induced in affected skin.

These genes are involved in the regulation of apoptosis and also partly in the regulation of cell proliferation.

FANCC regulates cell apoptosis induced by gamma-interferon (Pang Q, et al., EMBO J., (2001 August) 15;20(16):4478-89; Rathbun R K et al., Blood, (2000 December) 15;96(13):4204-11 Comment in: Blood, (2002 April) 1;99(7):2627-8; discussion 2629-30) and TNF-alpha (Pang Q, et al., EMBO J., (2001 August) 15;20(16):4478-89), which are two cytokines whose expression is increased in psoriasis (Chodorowska G., J. Eur. Acad. Dermatol. Venereol., (1998 March) 10(2):147-51; Uyemura K, Yamamura M, Fivenson D F et al., J. Invest. Dermatol., (1993 November) 101(5):701-5). FANCC also regulates apoptosis induced by oxidative stress or certain xenobiotics by modulating the activity of gluthatione-S-transferase P1 (GSTP1), responsible for the detoxification of these pro-apoptotic agents (Cumming R C et al., Nat. Med., (2001 July) 7(7):814-20 Comment in: Nat Med., (2001 December) 7(12):1259-60; Pagano G., Carcinogenesis, (2000 May) 21(5):1067-8). Now, oxidative stress has been described as one of the potential etiological factors for psoriasis, with in particular an increase in anti-oxidant activity in the disease (Shilov V N, Sergienko V I, Bull Exp. Biol. Med., (2000 April) 129(4):309-13). FANCC could therefore play a role in the mechanism involving oxidative stress in the disease.

In addition, FANCC plays a role in cell proliferation (Nadler J J, Braun R E., Genesis, (2000 July) 27(3):117-23; Haneline L S, Gobbett T A, Ramani R et al., Blood, (1999 July) 1;94(1):1-8).

DAD1 plays a role in apoptosis (Hong N A et al., Dev. Biol., (2000 April) 1;220(1):76-84; Nakashima T. et al., Mol. Cell Biol., (1993 October) 13(10):6367-74), and modulates the proliferation of T lymphocytes (Hong N A et al., J. Immunol., (1999 August) 15;163(4):1883-93), which are important cells in the physiopathology of psoriasis (Krueger J G, J. Am. Acad. Dermatol., (2002 January) 46(1):1-23, quiz 23-6).

GRIM19 is a modulator of apoptosis induced by beta-interferon and retinoic acid (Angell J E, Lindner D J, Shapiro P S et al., J. Biol. Chem., (2000 October) 27;275(43):33416-26).

HADHII, hydroxyacyl-CoA dehydrogenase type 11, is part of the short-chain dehydrogenase/reductase (SDR) family (Oppermann U C, Salim S et al., FEBS Lett, (1999 May) 28;451(3):238-42). HADHII is also involved in apoptosis and cell proliferation. Specifically, HADHII binds the A-beta amyloid peptide, which exerts regulatory effects on apoptosis. In addition, A-beta amyloid appears to modulate the cell proliferation, since its expression is associated with a senescent state of cells (Adler M J., Coronel C., Shelton E., Proc. Natl. Acad. Sci., USA (1991 January) 1;88(1):16-20).

In particular, the present invention relates to the use of at least one polynucleotide probe relating to the FANCC, DAD1, GRIM19 or HADHII gene as defined above, for diagnosing psoriasis or a predisposition to psoriasis by measuring the level of expression, respectively, of the FANCC, DAD1, GRIM19 OR HADHII gene.

The present invention also relates to the use of at least one polynucleotide probe relating to the FANCC, DAD1, GRIM19 or HADHII gene as defined above, for identifying a compound for use in the treatment of psoriasis.

In a first particular embodiment of the invention, the polynucleotide probe relating to the FANCC gene is a fragment of 10 to 500 base pairs representing at least 80%, and preferably at least 90%, homology with the single-stranded cDNA probe of sequence SEQ ID No. 2 or with the double-stranded cDNA probe of sequence SEQ ID No. 3 or with the RNA probe of sequence SEQ ID No. 4.

In a second particular embodiment of the invention, the polynucleotide probe relating to the DAD1 gene is a fragment of 10 to 500 base pairs representing at least 80%, and preferably at least 90%, homology with the single-stranded cDNA probe of sequence SEQ ID No. 6 or with the double-stranded cDNA probe of sequence SEQ ID No. 7 or with the RNA probe of sequence SEQ ID No. 8.

In a third particular embodiment of the invention, the polynucleotide probe relating to the GRIM19 gene is a fragment of 10 to 500 base pairs representing at least 80%, and preferably at least 90%, homology with the single-stranded cDNA probe of sequence SEQ ID No. 10 or with the double-stranded cDNA probe of sequence SEQ ID No. 11 or with the RNA probe of sequence SEQ ID No. 12.

In a fourth particular embodiment of the invention, the polynucleotide probe relating to the HADHII gene is a fragment of 10 to 500 base pairs representing at least 80%, and preferably at least 90%, homology with the single-stranded cDNA probe of sequence SEQ ID No. 14 or with the double-stranded cDNA probe of sequence SEQ ID No. 15 or with the RNA probe of sequence SEQ ID No. 16.

In another embodiment of the invention, the polynucleotide probe according to the invention will be used in the method described below.

The present invention thus features a method for identifying a compound for use in the treatment of psoriasis, comprising the following steps:

a) preparing at least two biological samples;

b) bringing one of the samples into contact with one or more compounds to be tested;

c) measuring the amount of messenger RNA corresponding to the FANCC and/or DAD1 and/or GRIM19 and/or HADHII gene in the biological samples using at least one polynucleotide probe, said probe having a sequence of 10 to 500 base pairs and being at least 80% complementary with a target sequence corresponding to all or part, respectively, of the FANCC gene (accession no. NM 000136.1) and/or DAD1 gene (accession no. NM 001344.1) and/or GRIM19 gene (accession no. NM 015965.3) and/or HADHII gene (accession no. NM 004493.1);

d) selecting said compounds for which a modulation of the transcription of the messenger RNAs of the FANCC gene and/or DAD1 gene and/or GRIM19 gene and/or HADHII gene or of the expression of the corresponding proteins is measured in the sample treated in step b).

The expression “modulation of the transcription of a gene or of the expression of the corresponding proteins” means an increase or a decrease in the amount of messenger RNAs or of the protein corresponding to said gene in a sample tested.

In particular, step c) of the method may be carried out with:

a polynucleotide probe relating to the FANCC gene, which is a fragment of 10 to 500 base pairs representing at least 80%, and preferably at least 90%, homology with the single-stranded cDNA probe of sequence SEQ ID No. 2 or with the double-stranded cDNA probe of sequence SEQ ID No. 3 or with the RNA probe of sequence SEQ ID No. 4;

a polynucleotide probe relating to the DAD1 gene, which is a fragment of 10 to 500 base pairs representing at least 80%, and preferably at least 90%, homology with the single-stranded cDNA probe of sequence SEQ ID No. 6 or with the double-stranded cDNA probe of sequence SEQ ID No. 7 or with the RNA probe of sequence SEQ ID No. 8;

a polynucleotide probe relating to the GRIM19 gene, which is a fragment of 10 to 500 base pairs representing at least 80%, and preferably at least 90%, homology with the single-stranded cDNA probe of sequence SEQ ID No. 10 or with the double-stranded cDNA probe of sequence SEQ ID No. 11 or with the RNA probe of sequence SEQ ID No. 12;

a polynucleotide probe relating to the HADHII gene, which is a fragment of 10 to 500 base pairs representing at least 80%, and preferably at least 90%, homology with the single-stranded cDNA probe of sequence SEQ ID No. 14 or with the double-stranded cDNA probe of sequence SEQ ID No. 15 or with the RNA probe of sequence SEQ ID No. 16.

According to the embodiment given in Example 1 to follow, the measurement of the expression of the FANCC and/or DAD1 and/or GRIM19 and/or HADHII gene(s) in a biological sample can be carried out using the technology consisting of cDNA arrays on a nylon support, according to which the mRNAs extracted from the biological sample are transcribed to radiolabeled single-stranded cDNAs, which are then deposited onto cDNA arrays.

According to another possible embodiment, the total mRNAs extracted from the biological sample tested can be analyzed according to one of the detection methods known to those skilled in the art (Sambrook et al., Molecular Cloning, A Laboratory Manual, Volume 1 (1989) Second Edition, chapter 7) such as the Northern blotting hybridization technique using a denatured double-stranded cDNA probe.

According to another possible embodiment, the probe labeling can comprise a cycle of double-stranded cDNA synthesis, from the total mRNAs extracted from the biological sample, coupled to in vitro transcription in the presence of biotinylated ribonucleotides, generating labeled complementary RNAs, these labeled antisense RNAs acting as a probe during hybridization on a DNA chip (Affymetrix, Gene Expression Monitoring, Technical Note; U.S. Pat. Nos. 5,716,785, 5,891,636 and 6,291,170).

In another embodiment, the present invention features the use of at least one polypeptide probe relating to the FANCC gene and/or DAD1 gene or/or GRIM19 gene and/or HADHII gene as defined above, for diagnosing psoriasis or a predisposition to psoriasis in a biological sample, by measuring the level of expression, respectively, of the FANCC gene and/or DAD1 gene or/or GRIM19 gene and/or HADHII gene.

The present invention also features the use of at least one polypeptide probe relating to the FANCC gene and/or DAD1 gene or/or GRIM19 gene and/or HADHII gene as defined above, for identifying a compound for use in the treatment of psoriasis.

The present invention also features a method for identifying a compound for use in the treatment of psoriasis, comprising the following steps:

a) preparing at least two biological samples;

b) bringing one of the samples into contact with one or more compounds to be tested;

c) measuring the amount of the protein produced by the expression of at least one gene selected from FANCC and/or DAD1 or/or GRIM19 and/or HADHII in the biological samples employing at least one polypeptide probe, said probe being a monoclonal or polyclonal antibody specifically directed against the wild-type protein derived from the expression, respectively, of the FANCC gene, DAD1 gene, GRIM19 gene or HADHII gene, or against a biologically active fragment of said protein, or else against a homologous, modified or variant polypeptide of the wild-type protein;

d) selecting said compounds for which a modulation of the expression of said protein(s) is measured in the sample treated in step b).

In another embodiment, the present invention features a research tool comprising a polynucleotide probe, said probe having a sequence of 10 to 500 base pairs and being at least 80% complementary with a target sequence corresponding to all or part of the FANCC gene (accession no. NM 000136.1) or DAD1 gene (accession no. NM 001344.1) or GRIM19 gene (accession no. NM 015965.3) or HADHII gene (accession no. NM 004493.1).

In particular, the polynucleotide probe is:

a polynucleotide probe relating to the FANCC gene, which is a fragment of 10 to 500 base pairs representing at least 80%, and preferably at least 90%, homology with the single-stranded cDNA probe of sequence SEQ ID No. 2 or with the double-stranded cDNA probe of sequence SEQ ID No. 3 or with the RNA probe of sequence SEQ ID No. 4;

a polynucleotide probe relating to the DAD1 gene, which is a fragment of 10 to 500 base pairs representing at least 80%, and preferably at least 90%, homology with the single-stranded cDNA probe of sequence SEQ ID No. 6 or with the double-stranded cDNA probe of sequence SEQ ID No. 7 or with the RNA probe of sequence SEQ ID No. 8;

a polynucleotide probe relating to the GRIM19 gene, which is a fragment of 10 to 500 base pairs representing at least 80%, and preferably at least 90%, homology with the single-stranded cDNA probe of sequence SEQ ID No. 10 or with the double-stranded cDNA probe of sequence SEQ ID No. 11 or with the RNA probe of sequence SEQ ID No. 12;

a polynucleotide probe relating to the HADHII gene, which is a fragment of 10 to 500 base pairs representing at least 80%, and preferably at least 90%, homology with the single-stranded cDNA probe of sequence SEQ ID No. 14 or with the double-stranded cDNA probe of sequence SEQ ID No. 15 or with the RNA probe of sequence SEQ ID No. 16.

The present invention also features a research tool comprising a polypeptide probe, said probe being a monoclonal or polyclonal antibody specifically directly against the wild-type protein derived from the expression of the FANCC gene or DAD1 gene or GRIM19 gene or HADHII gene, or against a biologically active fragment of said protein, or else against a homologous, modified or variant polypeptide of the wild-type protein.

The research tool may also make it possible to analyze the effects of certain treatments on the expression of these psoriasis marker genes, such as FANCC, DAD1, GRIM19 and HADHII, or to analyze mutation of the sequence of these genes in individuals suffering from psoriasis.

The research tool may also make it possible to analyze the expression profile of the FANCC gene and/or DAD1 gene and/or GRIM19 gene and/or HADHII gene in the cells constituting the epidermis, in particular the keratinocytes, for example according to their state of proliferation or of differentiation. The research tool may also allow the constitutive expression, the overexpression or the inhibition of the expression of the FANCC gene and/or DAD1 gene and/or GRIM19 gene and/or HADHII gene, according to in vitro or in vivo assays known to those skilled in the art.

For example, according to a research tool envisaged, the polynucleotide probe according to the invention can be used, for example, in a Northern blotting hybridization assay, or else in a PCR (polymerase chain reaction) amplification assay, so as to follow the expression of at least one gene selected from among FANCC, DAD1, GRIM19 and HADHII or of a homologous, variant, modified or partial sequence, in cells constituting the epidermis, in particular keratinocytes, expressing in vitro, at least one gene selected from among FANCC, DAD1, GRIM19 and HADHII, for example as a function of the state of differentiation or of proliferation of the cells. In particular, said expression may be constitutive or alternatively inducible, it being possible for the FANCC gene, DAD1 gene, GRIM19 gene or HADHII gene to be placed under the control of a constitutive promoter such as the SV40 or CMV promoter, or of an inducible promoter such as the Tet Off Gene Expression System (Invitrogen), conventional genetic engineering and transfection techniques are used (Gossen M, Bujard H., Annu. Rev. Genet., (2002) 36:153-73).

According to another research tool envisaged, the polynucleotide probe according to the invention can be used, for example in a Northern blotting hybridization assay, or else in a PCR (polymerase chain reaction) amplification assay, so as to follow the expression of the FANCC gene or DAD1 gene or GRIM19 gene or HADHII gene or of a homologous, variant, modified or partial sequence, in the cells constituting the epidermis of a laboratory animal strain, in particular in the keratinocytes, overexpressing, in vivo, said gene or sequence, for example under the control of a promoter specifically activated in the epidermis, such as the K14 or K15 keratin promoters or the involucrin promoter, for example as a function of the state of differentiation or of proliferation of the cells.

According to another research tool envisaged, the polynucleotide probe according to the invention can be used for the analysis, for example by Southern blotting, of an inactivation by deletion of the FANCC gene or DAD1 gene or GRIM19 gene or HADHII gene, for example in cells constituting the epidermis, in particular keratinocytes, cultured in vitro, or alternatively in vivo in the cells of a laboratory animal strain. This inactivation can be carried out according to conventional homologous recombination techniques.

According to another research tool envisaged, the polynucleotide probe according to the invention can be used for designing and synthesizing siRNAs, according to techniques known to those skilled in the art (Tuschl T, Borkhardt A, Mol. Interv., (2002) June;2(3):158-67). These antisense RNAs can be used for inhibiting the expression of the FANCC gene or DAD1 gene or GRIM19 gene or HADHII gene, for example in a line of cells constituting the epidermis, in particular keratinocytes, in vitro, or alternatively in vivo, for example in a laboratory animal strain.

The research tool may also make it possible to analyze the effects of certain treatments on the expression of these psoriasis marker genes, or to analyze mutation of the sequence of these genes in individuals suffering from psoriasis

For example, this research tool may be such that the probe(s) that it comprises is (are) brought into contact with the corresponding target sequence(s) deposited onto a support such as a nylon filter.

The probe can also be used in a hybridization assay (for example an assay for detecting mismatches), sequencing or microsequencing.

According to other research tools envisaged by the present invention, the probe according to the invention may be combined with another detectable probe, the nature of which may preferably be fluorescent, radioactive or enzymatic. This characteristic may make it possible, inter alia, to follow the localization of the probe, from the extracellular medium to the cell, or from the cytoplasm to the nucleus, or else to specify its interaction with DNA or RNA or proteins. Those skilled in the art will know which detectable probe is most suitable according to the characteristic that they wish to monitor.

According to a research tool envisaged, the polypeptide probe according to the invention can be used for immunolabeling of the target polypeptide as defined above, on tissue, according to conventional techniques known to those skilled in the art, for example on a section of epidermis from laboratory animals, for example the animals exhibiting overexpression of the FANCC protein or DAD1 protein or GRIM19 protein or HADHII protein or of a homologous, variant or modified polypeptide or active fragment of said protein, or else in vitro, on cells constituting the epidermis, in particular keratinocytes, that may be subject to overexpression of the protein or of said polypeptide, or else of inhibition of this expression, for example using siRNA as envisaged above.

According to another research tool envisaged, the polypeptide probe can also be used for purifying said protein or said polypeptide, for example according to immunoprecipitation techniques conventionally known to those skilled in the art.

The techniques conventionally known to those skilled in the art can be the techniques described by Sambrook J. Fritsch E F and Maniatis T in Molecular Cloning, A Laboratory Manual.

The discovery of the overexpression of FANCC, DAD1, GRIM19 and HADHII, in psoriatic skin, makes it possible, firstly, to diagnose psoriasis and, secondly, to develop novel therapies based on modulating the expression and/or the function of one or more of these genes, for treating psoriasis by acting on the apoptotic component with a view to limiting the cell proliferation disorders that in part characterize psoriasis.

In a particularly preferable embodiment, the present invention provides a method for characterizing skin samples in order to determine the predisposition to psoriasis.

It has now been demonstrated that the expression of the FANCC, DAD1, GRIM19 and HADHII genes is induced in lesional psoriatic skin exhibiting a psoriatic plaque, compared with normal skin (average expression measured in 13 psoriatic individuals and 10 normal individuals).

The results obtained are reported in Table 1.

Measurement of the expression of one of the genes, and preferably the four genes, FANCC, DAD1, GRIM19 and HADHII, makes it possible to characterize lesional psoriatic skin independently of the type and of the location of the psoriatic plaque (Tables 2 and 3). Table 2 represents the expression of the FANCC, DAD1, GRIM19 and HADHII genes, relative to an average expression in normal individuals, in patients exhibiting predominantly plaques on the trunk and asymmetric plaques. Table 3 represents the expression of the FANCC, DAD1, GRIM19 and HADHII genes, relative to an average expression in normal individuals, in patients exhibiting predominantly plaques of symmetric type.

In addition, besides the characterization of skin exhibiting a psoriatic plaque compared with normal skin, the discovery of these novel psoriasis markers also makes it possible to discriminate between a patient's psoriatic skin that is non-lesional (clinically normal) but has already entered into the psoriatic process, and non-lesional skin having the real characteristics of normal skin.

Thus, it has now been discovered, surprisingly, that some non-lesional skin also overexpresses the FANCC, DAD1, GRIM19 and HADHII genes. Based on the expression of these genes, the diagnosis of psoriasis can be determined very early since the expression of the FANCC, DAD1, GRIM19 and HADHII genes makes it possible to differentiate, at the molecular level, non-lesional skin that is already affected, whereas the clinical signs do not allow this. The results obtained are reported in Table 4.

Thus, the invention also features a method for diagnosing psoriasis or a predisposition to psoriasis, comprising a step entailing measuring the overexpression in a biological sample of at least one of the FANCC, DAD1, GRIM19 or HADHII genes, by bringing at least one polynucleotide probe into contact with the target sequences isolated from said biological sample, said probe having a sequence of 10 to 500 base pairs and being at least 80% complementary with a target sequence corresponding to all or part of the FANCC gene (accession no. NM 000136.1) or DAD1 gene (accession no. NM 001344.1) or GRIM19 gene (accession no. NM 015965.3) or HADHII gene (accession no. NM 004493.1).

In particular, the polynucleotide probe is:

a polynucleotide probe relating to the FANCC gene, which is a fragment of 10 to 500 base pairs representing at least 80%, and preferably at least 90%, homology with the single-stranded cDNA probe of sequence SEQ ID No. 2 or with the double-stranded cDNA probe of sequence SEQ ID No. 3 or with the RNA probe of sequence SEQ ID No. 4;

a polynucleotide probe relating to the DAD1 gene, which is a fragment of 10 to 500 base pairs representing at least 80%, and preferably at least 90%, homology with the single-stranded cDNA probe of sequence SEQ ID No. 6 or with the double-stranded cDNA probe of sequence SEQ ID No. 7 or with the RNA probe of sequence SEQ ID No. 8;

a polynucleotide probe relating to the GRIM19 gene, which is a fragment of 10 to 500 base pairs representing at least 80%, and preferably at least 90%, homology with the single-stranded cDNA probe of sequence SEQ ID No. 10 or with the double-stranded cDNA probe of sequence SEQ ID No. 11 or with the RNA probe of sequence SEQ ID No. 12;

a polynucleotide probe relating to the HADHII gene, which is a fragment of 10 to 500 base pairs representing at least 80%, and preferably at least 90%, homology with the single-stranded cDNA probe of sequence SEQ ID No. 14 or with the double-stranded cDNA probe of sequence SEQ ID No. 15 or with the RNA probe of sequence SEQ ID No. 16.

The present invention also features a method for diagnosing psoriasis or a predisposition to psoriasis, comprising a step entailing measuring the overexpression in a biological sample of the protein produced by the expression of the FANCC gene or DAD1 gene or GRIM19 gene or HADHII gene, by bringing into contact a polypeptide probe such as a monoclonal or polyclonal antibody specifically directed against the wild-type protein derived from the expression, respectively, of the FANCC gene or DAD1 gene or GRIM19 gene or HADHII gene, or against a biologically active fragment of said protein, or else against a homologous, modified or variant polypeptide of the wild-type protein.

The expression “overexpression of a gene in a biological sample” means expression of the messenger RNA or of the protein corresponding to this gene, that is higher in this sample compared with a reference normal sample.

Moreover, this invention also features a diagnostic kit comprising at least one polynucleotide or polypeptide probe relating to at least one of the FANCC, DAD1, GRIM19 and HADHII genes.

The present invention therefore features a diagnostic kit comprising at least one polynucleotide probe, said probe having a sequence of 10 to 500 base pairs and being at least 80% complementary with a target sequence corresponding to all or part of the FANCC gene (accession no. NM 000136.1) and/or DAD1 gene (accession no. NM 001344.1) and/or GRIM19 gene (accession no. NM 015965.3) and/or HADHII gene (accession no. NM 004493.1).

In particular, said polynucleotide probe of the diagnostic kit is:

a polynucleotide probe relating to the FANCC gene, which is a fragment of 10 to 500 base pairs representing at least 80%, and preferably at least 90%, homology with the single-stranded cDNA probe of sequence SEQ ID No. 2 or with the double-stranded cDNA probe of sequence SEQ ID No. 3 or with the RNA probe of sequence SEQ ID No. 4;

a polynucleotide probe relating to the DAD1 gene, which is a fragment of 10 to 500 base pairs representing at least 80%, and preferably at least 90%, homology with the single-stranded cDNA probe of sequence SEQ ID No. 6 or with the double-stranded cDNA probe of sequence SEQ ID No. 7 or with the RNA probe of sequence SEQ ID No. 8;

a polynucleotide probe relating to the GRIM19 gene, which is a fragment of 10 to 500 base pairs representing at least 80%, and preferably at least 90%, homology with the single-stranded cDNA probe of sequence SEQ ID No. 10 or with the double-stranded cDNA probe of sequence SEQ ID No. 11 or with the RNA probe of sequence SEQ ID No. 12;

a polynucleotide probe relating to the HADHII gene, which is a fragment of 10 to 500 base pairs representing at least 80%, and preferably at least 90%, homology with the single-stranded cDNA probe of sequence SEQ ID No. 14 or with the double-stranded cDNA probe of sequence SEQ ID No. 15 or with the RNA probe of sequence SEQ ID No. 16.

The present invention also features a diagnostic kit comprising at least one polypeptide probe such as a monoclonal or polyclonal antibody specifically directed against the wild-type protein derived from the expression of the FANCC gene or DAD1 gene or GRIM19 gene or HADHII gene, or against a biologically active fragment of said protein, or else against a homologous, modified or variant polypeptide of the wild-type protein.

The observations described above also indicate that FANCC, DAD1, GRIM19 and HADHII are therapeutic targets for the development of novel therapies suited for treating chronic plaque-type psoriasis vulgaris, whatever the predominant type of plaques affecting the patient, and the location of the psoriatic plaque on the body.

Thus, the present invention features the use of a polynucleotide probe or of one of its analogues, complementary, respectively, to the FANCC gene or DAD1 gene or GRIM19 gene or HADHII gene, for modulating, respectively, the expression of the FANCC gene or DAD1 gene or GRIM19 gene or HADHII gene.

In particular, the polynucleotide probe may be an antisense RNA or an siRNA, the role of which is to inhibit the expression of a specific gene (Fire A., Trends in Genetic, (1999) 15:358-363).

In a particular embodiment of the invention, the polynucleotide probe is:

a polynucleotide probe relating to the FANCC gene, which is a fragment of 10 to 500 base pairs representing at least 80%, and preferably at least 90%, homology with the RNA probe of sequence SEQ ID No. 4;

a polynucleotide probe relating to the DAD1 gene, which is a fragment of 10 to 500 base pairs representing at least 80%, and preferably at least 90%, homology with the RNA probe of sequence SEQ ID No. 8;

a polynucleotide probe relating to the GRIM19 gene, which is a fragment of 10 to 500 base pairs representing at least 80%, and preferably at least 90%, homology with the RNA probe of sequence SEQ ID No. 12;

a polynucleotide probe relating to the HADHII gene, which is a fragment of 10 to 500 base pairs representing at least 80%, and preferably at least 90%, homology with the RNA probe of sequence SEQ ID No. 16.

Finally, the invention also features the use of at least one polynucleotide probe that has a sequence of 10 to 500 base pairs and is at least 80% complementary with a target sequence corresponding to all or part of the FANCC gene (accession no. NM 000136.1) and/or DAD1 gene (accession no. NM 001344.1) and/or GRIM19 gene (accession no. NM 015965.3) and/or HADHII gene (accession no. NM 004493.1), for the manufacture of a pharmaceutical composition for use in the treatment of psoriasis.

The polynucleotide probe may more particularly be:

a polynucleotide probe relating to the FANCC gene, which is a fragment of 10 to 500 base pairs representing at least 80%, and preferably at least 90%, homology with the single-stranded cDNA probe of sequence SEQ ID No. 2 or with the double-stranded cDNA probe of sequence SEQ ID No. 3 or with the RNA probe of sequence SEQ ID No. 4;

a polynucleotide probe relating to the DAD1 gene, which is a fragment of 10 to 500 base pairs representing at least 80%, and preferably at least 90%, homology with the single-stranded cDNA probe of sequence SEQ ID No. 6 or with the double-stranded cDNA probe of sequence SEQ ID No. 7 or with the RNA probe of sequence SEQ ID No. 8;

a polynucleotide probe relating to the GRIM19 gene, which is a fragment of 10 to 500 base pairs representing at least 80%, and preferably at least 90%, homology with the single-stranded cDNA probe of sequence SEQ ID No. 10 or with the double-stranded cDNA probe of sequence SEQ ID No. 11 or with the RNA probe of sequence SEQ ID No. 12;

a polynucleotide probe relating to the HADHII gene, which is a fragment of 10 to 500 base pairs representing at least 80%, and preferably at least 90%, homology with the single-stranded cDNA probe of sequence SEQ ID No. 14 or with the double-stranded cDNA probe of sequence SEQ ID No. 15 or with the RNA probe of sequence SEQ ID No. 16.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Measurement of Gene Expression in Normal Skin and in Psoriatic Skin

A clinical study carried out in the presence of normal volunteers and of patients suffering from chronic plaque-type psoriasis vulgaris made it possible to demonstrate the involvement of the FANCC, DAD1, GRIM19 and HADHII genes in psoriasis. Skin biopsies were taken from normal volunteers, and also from psoriatic plaques and from non-lesional regions that appeared to be clinically normal, in patients suffering from psoriasis. The presence or absence of psoriasis on the biopsied skin region, and also the severity of the psoriatic plaque, were determined by evaluating local clinical parameters (erythema score, desquamation score and psoriatic plaque elevation score).

The gene expression analysis is then carried out using each individual biopsy. Comparison of the gene profiles corresponding to the normal skin and the non-lesional and lesional psoriatic skin makes it possible to identify genes whose expression is induced in the disease.

The gene profile determination is based on the hybridization of a complex single-stranded cDNA probe (generated by reverse transcription of the mRNAs derived from each skin biopsy) on cDNA fragments of interest, which are deposited onto a support such as a nylon filter. The hybridization signals obtained are proportional to the amount of each labeled cDNA in the probe and reflect the level of expression of each mRNA within the skin sample analyzed (Nguyen C et al., Genomics, (1995 September) 1;29(1):207-16).

Dermatology-specific microarray filters produced by the inventors were developed. These microarray filters consist of 474 cDNAs involved in the physiological processes of human skin. In parallel, commercial microarray filters comprising 4200 human cDNAs (Invitrogen GF211) were used. These cDNA microarray filters will make it possible to determine the expression of genes of interest in normal skin and also in non-lesional and lesional skin from patients suffering from chronic plaque-type psoriasis vulgaris.

Method:

Gene expression in normal skin and in psoriatic skin is carried out using the technology of cDNA microarray filters on a nylon support.

The principle of this technology is based on the hybridization of a radiolabeled single-stranded cDNA probe on target cDNA fragments deposited at high density onto nylon filters (Nguyen C et al., Genomics, (1995 September) 1;29(1):207-16). In this study, nylon microarray filters (GF211) sold by the company Invitrogen, onto which 4200 identified human cDNAs are deposited, and also dermatology-specific filters comprising 474 human cDNAs, developed by the inventors, are used. The radiolabeled cDNA probes are generated by reverse transcription of the total RNAs (Invitrogen protocol) isolated from the various biopsies taken from 10 normal volunteers and 13 individuals suffering from chronic plaque-type psoriasis vulgaris (RNEasy protocol, Qiagen). The hybridization signals obtained are proportional to the amount of each labeled cDNA present in the probe, which is hybridized to the cDNA complementary thereto on the microarray filter, and thus reflect the transcriptional activity of the mRNAs in the biopsies analyzed.

Normalization Analysis of the Gene Expression Results and Comparison of the Gene Profiles Between Normal Skin and Psoriatic Skin:

The hybridization signals are measured by phospholuminescence, and then quantified using the Imagen analysis program (Biodiscovery).

In order to discriminate the hybridization signals specific for the genes studied, the background noise associated with non-specific hybridization is measured on each microarray filter on blank areas of cDNA deposits (GF211 microarray filters), or in a ring surrounding each spot (specific microarray filters). After conversion of the data to log values, a detection threshold for discriminating a measurement that comes from a real signal or from the noise is determined automatically for each filter using the method described previously by Fogel et al., 2002.

With a view to comparing the results of gene expression between the various biopsies analyzed, the hybridization signals are normalized by means of the median of all signals obtained for each biopsy (GF211 microarray filters) or by means of the expression of an exogenous messenger RNA, acting as a reference, the expression of which does not vary between the samples analyzed. This exogenous transcript is added in constant amount to the total RNA preparations derived from the various samples, before synthesis of the radiolabeled cDNA probe. The target sequence for this exogenous transcript is deposited onto each microarray filter.

For each gene, a reference average expression is determined on all the biopsies from normal individuals, and then subtracted from the expression obtained in each biopsy of non-lesional and lesional psoriatic skin (values in log). The ratio of the expression of each gene in the psoriatic skin to the expression in normal skin is then obtained by taking the base 10 exponential of the value previously calculated (Tables 1 to 4).

The results obtained make it possible to demonstrate the overexpression of the FANCC, DAD1, GRIM19 and HADHII genes in lesional human skin exhibiting a psoriatic plaque, compared with normal human skin (Table 1).

The results in Tables 2 and 3 indicate that these overexpressions are observed whatever the predominant type of plaques expressed in the patient (symmetrical or on the trunk) and also independently of the location of the psoriatic plaques. The abbreviations mentioned in these tables represent the location of the biopsies taken on the body for each psoriatic individual (PI) and have the following meaning: LL: left leg, RL: right leg, RA: right arm, LA: left arm, T: trunk.

The results in Table 4 show that the expression of these genes is also induced in non-lesional psoriatic skin. The abbreviations have the same meaning as above.

### EXAMPLE 2

### Measurements of Local Clinical Parameters Associated with Plaque-Type Psoriasis: Erythema Score, Desquamation Score and Plaque Elevation Score:

The various local clinical scores for defining the presence or absence of psoriasis on the biopsied skin region, and also the severity of the psoriatic plaque, are the erythema score, the desquamation score and the plaque elevation score. These scores are conventionally measured by means of the following classifications:

Erythema is measured using the following classification:

0=none (no erythema)

1=mild (mild redness present)

2=moderate (readily detectable redness)

3 severe (intense redness)

4=very severe (very intense redness)

Desquamation is measured using the following classification:

0=none (no desquamation)

1=mild (squamae detected, a single layer of desquamation)

2=moderate (squamae of intermediate degree between mild and severe)

3=severe (several layers of squamae)

4=very severe (several layers of thick squamae)

Plaque elevation is measured using the following classification:

0=none (no elevation of thickness)

1=mild (mild elevation detectable to the touch)

2=moderate (moderate thickness)

3=severe (substantial thickening)

4=very severe (very substantial thickening)

This study demonstrates that the local clinical parameters obtained with non-lesional psoriatic skin are not different from those obtained for normal skin. They do not therefore make it possible to differentiate between a non-lesional psoriatic skin and a normal skin.

On the other hand, the results in Table 4 made it possible to demonstrate, at the molecular level, an overexpression of the FANCC, DAD1, GRIM19 and HADHII genes in some non-lesional psoriatic skin.

Measurement of the expression of the FANCC, DAD1, GRIM19 and HADHII genes therefore makes it possible to determine a very early diagnosis of psoriasis, since the expression of these genes makes it possible to differentiate, at the molecular level, non-lesional skin that is already affected, whereas the clinical signs do not allow this.

TABLE 1

| | Average expression in normal | Average expression in |
|---|---|---|
| Name of gene | individuals | psoriatic individuals |
| FANCC | 1.0 | 2.6 |
| GRIM19 | 1.0 | 2.3 |
| DAD1 | 1.0 | 2.0 |
| HADHII | 1.0 | 1.9 |

TABLE 2

| | Biopsy | | | | | |
|---|---|---|---|---|---|---|
| | | | | | | PI99- |
| | PI54-T | PI54-LA | PI29-T | PI29-RA | PI99-T | RA |
| FANCC | 1.7 | 2.7 | 2.6 | 4.3 | 6.3 | 4.8 |
| GRIM19 | 1.9 | 1.8 | 4.3 | 4.5 | 6.9 | 3.1 |
| DAD1 | 1.2 | 1.0 | 1.5 | 1.8 | 4.0 | 3.2 |
| HADHII | 2.9 | 3.5 | 4.2 | 4.9 | 2.2 | 1.9 |

TABLE 3

| | Biopsies | | | | | | | | | | | | | | | | |
|---|---|---|---|---|---|---|---|---|---|---|---|---|---|---|---|---|---|
| | PI72 | PI72- | PI72- | PI100- | PI100- | PI100- | PI31- | PI31- | PI31- | PI28- | PI28 | PS96- | PI96- | PI96- | PI93- | PI93- | PI93- |
| | T | LL | RL | T | LL | RL | T | LA | RA | T | RL | T | LL | RL | T | LA | RA |
| FANCC | 1.3 | 2.4 | 2.3 | 1.3 | 1.9 | 1.4 | 5.0 | 5.2 | 2.7 | 3.4 | 3.2 | 3.8 | 2.7 | 3.7 | 2.8 | 8.5 | 5.9 |
| GRIM19 | 0.4 | 0.4 | 0.9 | 3.4 | 2.6 | 3.9 | 3.7 | 2.7 | 3.2 | 1.0 | 1.2 | 4.0 | 3.4 | 6.1 | 4.2 | 6.9 | 6.2 |
| DAD1 | 4.2 | 4.9 | 5.0 | 1.1 | 0.9 | 1.0 | 1.9 | 1.9 | 1.7 | 2.5 | 2.4 | 3.2 | 2.8 | 3.5 | 3.4 | 5.3 | 3.4 |
| HADHII | 0.9 | 1.1 | 1.2 | 1.3 | 1.7 | 1.2 | 4.1 | 3.9 | 3.8 | 1.3 | 1.3 | 1.3 | 1.1 | 2.1 | 2.3 | 4.2 | 3.5 |

TABLE 4

| | Average | Expression in clinically normal (non- | | | |
|---|---|---|---|---|---|
| | expression in | lesional) skin of psoriatic individuals | | | |
| Name | normal | PI96NL- | PI93NL- | PI72NL- | PI99NL- |
| of gene | individuals | RL | LA | LL | T |
| FANCC | 1.00 | 1.0 | 1.4 | 1.6 | 2.1 |
| GRIM19 | 1.0 | 1.9 | 1.9 | 0.4 | 1.1 |
| DAD1 | 1.0 | 2.1 | 2.6 | 4.1 | 3.4 |
| HADHII | 1.0 | 1.0 | 1.4 | 1.1 | 1.9 |
| | Average of 3 local clinical scores: | | | | |
| | 0 | 0 | 0 | 0 | 0 |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A polynucleotide probe having a sequence of 10 to 500 base pairs and being at least 80% complementary with a target sequence corresponding to all or part of a gene selected from the group consisting of FANCC (accession no. NM 000136.1), DAD1 (accession no. NM 001344.1), GRIM19 (accession no. NM 015965.3) and HADHII (accession no. NM 004493.1).

2. The polynucleotide probe as defined by claim 1, having a sequence of 10 to 500 base pairs and being at least 90% complementary with a target sequence corresponding to all or part of a gene selected from the group consisting of FANCC (accession no. NM 000136.1), DAD1 (accession no. NM 001344.1), GRIM19 (accession no. NM 015965.3) and HADHII (accession no. NM 004493.1).

3. An isolated polynucleotide 10 to 500 nucleotides in length, wherein said polynucleotide is at least 80% complementary to the same number of nucleotides of a sequence selected from the group consisting of SEQ ID NO:2, SEQ ID NO:4, SEQ ID NO:6, SEQ ID NO:8, SEQ ID NO:10, SEQ ID NO:12, SEQ ID NO:14, and SEQ ID NO:16.

4. The isolated polynucleotide as defined by claim 3, wherein said polynucleotide is at least 90% complementary to the same number of nucleotides of a sequence selected from the group consisting of SEQ ID NO:2, SEQ ID NO:4, SEQ ID NO:6, SEQ ID NO:8, SEQ ID NO:10, SEQ ID NO:12, SEQ ID NO:14, and SEQ ID NO:16.

5. The isolated polynucleotide as defined by claim 3, wherein said polynucleotide is at least 95% complementary to the same number of nucleotides of a sequence selected from the group consisting of SEQ ID NO:2, SEQ ID NO:4, SEQ ID NO:6, SEQ ID NO:8, SEQ ID NO:10, SEQ ID NO:12, SEQ ID NO:14, and SEQ ID NO:16.

6. The isolated polynucleotide as defined by claim 3, wherein said polynucleotide is 100% complementary to the same number of nucleotides of a sequence selected from the group consisting of SEQ ID NO:2, SEQ ID NO:4, SEQ ID NO:6, SEQ ID NO:8, SEQ ID NO:10, SEQ ID NO:12, SEQ ID NO:14, and SEQ ID NO:16.

7. An isolated polynucleotide 10 to 500 nucleotides in length, wherein said polynucleotide is at least 80% complementary to the same number of nucleotides of one strand of the sequence selected from the group consisting of SEQ ID NO:3, SEQ ID NO:7, SEQ ID NO:11, and SEQ ID NO:15.

8. An isolated polynucleotide 10 to 500 nucleotides in length, wherein said polynucleotide is at least 90% complementary to the same number of nucleotides of one strand of the sequence selected from the group consisting of SEQ ID NO:3, SEQ ID NO:7, SEQ ID NO:11, and SEQ ID NO:15.

9. An isolated polynucleotide 10 to 500 nucleotides in length, wherein said polynucleotide is at least 95% complementary to the same number of nucleotides of one strand of the sequence selected from the group consisting of SEQ ID NO:3, SEQ ID NO:7, SEQ ID NO:11, and SEQ ID NO:15.

10. An isolated polynucleotide 10 to 500 nucleotides in length, wherein said polynucleotide is at least 100% complementary to the same number of nucleotides of one strand of the sequence selected from the group consisting of SEQ ID NO:3, SEQ ID NO:7, SEQ ID NO:11, and SEQ ID NO:15.

11. An expression vector comprising the isolated polynucleotide as defined by claim 3.

12. A cultured host cell comprising the expression vector as defined by claim 11.

13. A method of producing a polypeptide, said method comprising the steps of:
(a) culturing a host cell comprising the expression vector as defined by claim 11, and
(b) isolating the polypeptide expressed from said host cell.

14. A method for identifying a compound useful in the treatment of psoriasis, comprising:
(a) preparing at least two biological samples;
(b) bringing one of said samples into contact with one or more candidate compounds;
(c) measuring the amount of mRNA corresponding to the FANCC gene in said biological samples using at least one polynucleotide probe, wherein said probe is an isolated polynucleotide 10 to 500 nucleotides in length, wherein said polynucleotide is at least 80% complementary to the same number of nucleotides in SEQ ID NO:2 or SEQ ID NO:4; and
(d) selecting said compound for which a modulation of the transcription of the mRNAs of the FANCC gene or corresponding protein is measured in the sample treated in step (b).

15. The method as defined by claim 14, wherein said polynucleotide is at least 90% complementary to the same number of nucleotides in SEQ ID NO:2 or SEQ ID NO:4.

16. The method as defined by claim 14, wherein said polynucleotide is at least 95% complementary to the same number of nucleotides in SEQ ID NO:2 or SEQ ID NO:4.

17. The method as defined by claim 14, wherein said polynucleotide is at least 100% complementary to the same number of nucleotides in SEQ ID NO:2 or SEQ ID NO:4.

18. A method for identifying a compound useful in the treatment of psoriasis, comprising:
(a) preparing at least two biological samples;
(b) bringing one of said samples into contact with one or more candidate compounds;
(c) measuring the amount of mRNA corresponding to the DAD1 gene in said biological samples using at least one polynucleotide probe, wherein said probe is an isolated polynucleotide 10 to 500 nucleotides in length, wherein said polynucleotide is at least 80% complementary to the same number of nucleotides in SEQ ID NO:6 or SEQ ID NO:8; and
(d) selecting said compound for which a modulation of the transcription of the mRNAs of the DAD1 gene or corresponding protein is measured in the sample treated in step (b).

19. The method as defined by claim 18, wherein said polynucleotide is at least 90% complementary to the same number of nucleotides in SEQ ID NO:6 or SEQ ID NO:8.

20. The method as defined by claim 18, wherein said polynucleotide is at least 95% complementary to the same number of nucleotides in SEQ ID NO:6 or SEQ ID NO:8.

21. The method as defined by claim 18, wherein said polynucleotide is at least 100% complementary to the same number of nucleotides in SEQ ID NO:6 or SEQ ID NO:8.

22. A method for identifying a compound useful in the treatment of psoriasis, comprising:
(a) preparing at least two biological samples;
(b) bringing one of said samples into contact with one or more candidate compounds;
(c) measuring the amount of mRNA corresponding to the GRIM19 gene in said biological samples using at least one polynucleotide probe, wherein said probe is an isolated polynucleotide 10 to 500 nucleotides in length, wherein said polynucleotide is at least 80% complementary to the same number of nucleotides in SEQ ID NO:10 or SEQ ID NO:12; and
(d) selecting said compound for which a modulation of the transcription of the mRNAs of the GRIM19 gene or corresponding protein is measured in the sample treated in step (b).

23. The method as defined by claim 22, wherein said polynucleotide is at least 90% complementary to the same number of nucleotides in SEQ ID NO:10 or SEQ ID NO:12.

24. The method as defined by claim 22, wherein said polynucleotide is at least 95% complementary to the same number of nucleotides in SEQ ID NO:10 or SEQ ID NO:12.

25. The method as defined by claim 22, wherein said polynucleotide is at least 100% complementary to the same number of nucleotides in SEQ ID NO:10 or SEQ ID NO:12.

26. A method for identifying a compound useful in the treatment of psoriasis, comprising:
(a) preparing at least two biological samples;
(b) bringing one of said samples into contact with one or more candidate compounds;
(c) measuring the amount of mRNA corresponding to the HADHII gene in said biological samples using at least one polynucleotide probe, wherein said probe is an isolated polynucleotide 10 to 500 nucleotides in length, wherein said polynucleotide is at least 80% complementary to the same number of nucleotides in SEQ ID NO:14 or SEQ ID NO:16; and
(d) selecting said compound for which a modulation of the transcription of the mRNAs of the HADHII gene or corresponding protein is measured in the sample treated in step (b).

27. The method as defined by claim 26, wherein said polynucleotide is at least 90% complementary to the same number of nucleotides in SEQ ID NO:14 or SEQ ID NO:16.

28. The method as defined by claim 26, wherein said polynucleotide is at least 95% complementary to the same number of nucleotides in SEQ ID NO:14 or SEQ ID NO:16.

29. The method as defined by claim 26, wherein said polynucleotide is at least 100% complementary to the same number of nucleotides in SEQ ID NO:14 or SEQ ID NO:16.

30. An isolated monoclonal antibody that selectively binds to a protein, or a biologically-active fragment thereof, encoded by the amino acid sequence produced from a sequence selected from the group consisting of: SEQ ID NO:2, SEQ ID NO:4, SEQ ID NO:6, SEQ ID NO:8, SEQ ID NO:10, SEQ ID NO:12, SEQ ID NO:14, and SEQ ID NO:16.

31. A hybridoma cell line capable of producing the monoclonal antibody as defined by claim 30.

32. A composition comprising a peptide encoded by the amino acid sequence produced from a sequence selected from the group consisting of:
SEQ ID NO:2, SEQ ID NO:4, SEQ ID NO:6, SEQ ID NO:8, SEQ ID NO:10, SEQ ID NO:12, SEQ ID NO:14, and SEQ ID NO:16.

33. A method for ameliorating psoriasis, comprising administering to a patient suffering from psoriasis a therapeutically-effective amount of a compound identified via the method defined by claim 14.

34. A method for ameliorating psoriasis, comprising administering to a patient suffering from psoriasis a therapeutically-effective amount of a compound identified via the method defined by claim 18.

35. A method for ameliorating psoriasis, comprising administering to a patient suffering from psoriasis a therapeutically-effective amount of a compound identified via the method defined by claim 22.

36. A method for ameliorating psoriasis, comprising administering to a patient suffering from psoriasis a therapeutically-effective amount of a compound identified via the method defined by claim 26.


SEQUENCE LISTING (16 sequences)

SEQ ID NO: 1 - RNA, 500 nt, Homo sapiens
agacagguggcggccgggcaggcacucuuaagcccaccucccccucuuguugccuucgauuucggcaaagccugggcaggugccaccgggaaggaauggcaucgagaugcugggcggggacgcggcguggcgagggggcuugacggcguuggcggggcugggcacaggggcagccgcagggaggcagggauggcaaggcgugaagccacccuggaaggaacuggaccaaggucuucagaggugcgacagggucuggaaucugaccuuacucuagcaggaguuuuuguagacucucccugauaguuuaguuuuugauaaagcaugcugguaaaaccacuacccucagagagagccaaaaauacagaagaggcggagagcgccccuccaaccaggcuguuauuccccuggacuccgugacaucuguggaauuuuuuagcucuuuaaaaucuguaauuuguugucuauuuuuucauucuaaauaaaacuucaguuugcaccua

SEQ ID NO: 2 - DNA, 500 bp, Homo sapiens
taggtgcaaactgaagttttatttagaatgaaaaaatagacaacaaattacagattttaaagagctaaaaaattccacagatgtcacggagtccaggggaataacagcctggttggaggggcgctctccgcctcttctgtatttttggctctctctgagggtagtggttttaccagcatgctttatcaaaaactaaactatcagggagagtctacaaaaactcctgctagagtaaggtcagattccagaccctgtcgcacctctgaagaccttggtccagttccttccagggtggcttcacgccttgccatccctgcctccctgcggctgcccctgtgcccagccccgccaacgccgtcaagccccctcgccacgccgcgtccccgcccagcatctcgatgccattccttcccggtggcacctgcccaggctttgccgaaatcgaaggcaacaagagggggaggtgggcttaagagtgcctgcccggccgccacctgtct

SEQ ID NO: 3 - DNA, 500 bp, Homo sapiens
agacaggtggcggccgggcaggcactcttaagcccacctccccctcttgttgccttcgatttcggcaaagcctgggcaggtgccaccgggaaggaatggcatcgagatgctgggcggggacgcggcgtggcgagggggcttgacggcgttggcggggctgggcacaggggcagccgcagggaggcagggatggcaaggcgtgaagccaccctggaaggaactggaccaaggtcttcagaggtgcgacagggtctggaatctgaccttactctagcaggagtttttgtagactctccctgatagtttagtttttgataaagcatgctggtaaaaccactaccctcagagagagccaaaaatacagaagaggcggagagcgcccctccaaccaggctgttattcccctggactccgtgacatctgtggaattttttagctctttaaaatctgtaatttgttgtctattttttcattctaaataaaacttcagtttgcaccta

SEQ ID NO: 4 - RNA, 500 nt, Homo sapiens
uaggugcaaacugaaguuuuauuuagaaugaaaaaauagacaacaaauuacagauuuuauagagcuaaaaaauuccacagaugucacggaguccaggggaauaacagccugguuggaggggcgcucuccgccucuucuguauuuuuggcucucucugaggguagugguuuuaccagcaugcuuuaucaaaaacuaaacuaucagggagagucuacaaaaacuccugcuagaguaaggucagauuccagacccugucgcaccucugaagaccuugguccaguuccuuccaggguggcuucacgccuugccaucccugccucccugcggcugccccugugcccagccccgccaacgccgucaagcccccucgccacgccgcguccccgcccagcaucucgaugccauuccuucccgguggcaccugcccaggcuuugccgaaaucgaaggcaacaagagggggaggugggcuuaagagugccugcccggccgccaccugucu

SEQ ID NO: 5 - RNA, 500 nt, Homo sapiens
gucuccucguggggaccuuccccuucaacucuuuucucucgggcuucaucucuuguguggggaguuucauccuagcgguuugccugagaauacagaucaacccacagaacaaagcggauuuccaaggcaucuccccagagcgagccuuugcugauuuucucuuugccagcaccauccugcaccuuguugucaugaacuuuguuggcugaaucauucucauuuacuuaauugaggaguaggagacuaaaagaauguucacucuuugaauuuccuggauaagaguucuggagauggcagcuuauuggacacauggauuuucuucagauuugacacuuacugcuagcucugcuuuuuaugacaggagaaaagcccagaguucacugugugucagaacaacuuucuaacaaacauuuauuaauccagccucugccuuucauuaaauguaaccuuuugcuuuccaaauuaaagaacuccaugccacuccucaaaaaaaaaaaaaa

SEQ ID NO: 6 - DNA, 500 bp, Homo sapiens
ttttttttttttttgaggagtggcatggagttctttaatttggaaagcaaaaggttacatttaatgaaaggcagaggctggattaataaatgtttgttagaaagttgttctgacacacagtgaactctgggcttttctcctgtcataaaaagcagagctagcagtaagtgtcaaatctgaagaaaatccatgtgtccaataagctgccatctccagaactcttatccaggaaattcaaagagtgaacattcttttagtctcctactcctcaattaagtaaatgagaatgattcagccaacaaagttcatgacaacaaggtgcaggatggtgctggcaaagagaaaatcagcaaaggctcgctctggggagatgccttggaaatccgctttgttctgtgggttgatctgtattctcaggcaaaccgctaggatgaaactccccacacaagagatgaagcccgagagaaaagagttgaaggggaaggtccccacgaggagac

SEQ ID NO: 7 - DNA, 500 bp, Homo sapiens
gtctcctcgtggggaccttccccttcaactcttttctctcgggcttcatctcttgtgtggggagtttcatcctagcggtttgcctgagaatacagatcaacccacagaacaaagcggatttccaaggcatctccccagagcgagcctttgctgattttctctttgccagcaccatcctgcaccttgttgtcatgaactttgttggctgaatcattctcatttacttaattgaggagtaggagactaaaagaatgttcactctttgaatttcctggataagagttctggagatggcagcttattggacacatggattttcttcagatttgacacttactgctagctctgctttttatgacaggagaaaagcccagagttcactgtgtgtcagaacaactttctaacaaacatttattaatccagcctctgcctttcattaaatgtaaccttttgctttccaaattaaagaactccatgccactcctcaaaaaaaaaaaaaa

SEQ ID NO: 8 - RNA, 500 nt, Homo sapiens
uuuuuuuuuuuuuugaggaguggcauggaguucuuuaauuuggaaagcaaaagguuacauuuaaugaaaggcagaggcuggauuaauaaauguuuguuagaaaguuguucugacacacagugaacucugggcuuuucuccugucauaaaaagcagagcuagcaguaagugucaaaucugaagaaaauccauguguccaauaagcugccaucuccagaacucuuauccaggaaauucaaagagugaacauucuuuuagucuccuacuccucaauuaaguaaaugagaaugauucagccaacaaaguucaugacaacaaggugcaggauggugcuggcaaagagaaaaucagcaaaggcucgcucuggggagaugccuuggaaauccgcuuuguucuguggguugaucuguauucucaggcaaaccgcuaggaugaaacuccccacacaagagaugaagcccgagagaaaagaguugaaggggaagguccccacgaggagac

SEQ ID NO: 9 - RNA, 499 nt, Homo sapiens
aacggaacuugccgcgucgaggacugucgggcuacagcaugcuggccauagggauuggaacccugaucuacgggcacuggagcauaaugaaguggaaccgugagcgcaggcgccuacaaaucgaggacuucgaggcucgcaucgcgcuguugccacuguuacaggcagaaaccgaccggaggaccuugcagaugcuucgggagaaccuggaggaggaggccaucaucaugaaggacgugcccgacuggaaggugggggagucuguguuccacacaacccgcugggugccccccuugaucggggagcuguacgggcugcgcaccacagaggaggcucuccaugccagccacggcuucaugugguacacguaggcccugugcccuccggccaccuggaucccugccccuccccacugggacggaauaaaugcucugcagaccuggaaaaaaaaaaaaaaaaaaaaaaaaaaaaaaaaaaaaaaaaaaaaaaaaaaaaaaaa

SEQ ID NO: 10 - DNA, 499 bp, Homo sapiens
ttttttttttttttttttttttttttttttttttttttttttttttttttttttttccaggtctgcagagcatttattccgtcccagtggggaggggcagggatccaggtggccggagggcacagggcctacgtgtaccacatgaagccgtggctggcatggagagcctcctctgtggtgcgcagcccgtacagctccccgatcaaggggggcacccagcgggttgtgtggaacacagactcccccaccttccagtcgggcacgtccttcatgatgatggcctcctcctccaggttctcccgaagcatctgcaaggtcctccggtcggtttctgcctgtaacagtggcaacagcgcgatgcgagcctcgaagtcctcgatttgtaggcgcctgcgctcacggttccacttcattatgctccagtgcccgtagatcagggttccaatccctatggccagcatgctgtagcccgacagtcctcgacgcggcaagttccgtt

SEQ ID NO: 11 - DNA, 499 bp, Homo sapiens
aacggaacttgccgcgtcgaggactgtcgggctacagcatgctggccatagggattggaaccctgatctacgggcactggagcataatgaagtggaaccgtgagcgcaggcgcctacaaatcgaggacttcgaggctcgcatcgcgctgttgccactgttacaggcagaaaccgaccggaggaccttgcagatgcttcgggagaacctggaggaggaggccatcatcatgaaggacgtgcccgactggaaggtgggggagtctgtgttccacacaacccgctgggtgccccccttgatcggggagctgtacgggctgcgcaccacagaggaggctctccatgccagccacggcttcatgtggtacacgtaggccctgtgccctccggccacctggatccctgcccctccccactgggacggaataaatgctctgcagacctggaaaaaaaaaaaaaaaaaaaaaaaaaaaaaaaaaaaaaaaaaaaaaaaaaaaaaaaa

SEQ ID NO: 12 - RNA, 499 nt, Homo sapiens
uuuuuuuuuuuuuuuuuuuuuuuuuuuuuuuuuuuuuuuuuuuuuuuuuuuuuuuuccaggucugcagagcauuuauuccgucccaguggggaggggcagggauccagguggccggagggcacagggccuacguguaccacaugaagccguggcuggcauggagagccuccucuguggugcgcagcccguacagcuccccgaucaaggggggcacccagcggguuguguggaacacagacucccccaccuuccagucgggcacguccuucaugaugauggccuccuccuccagguucucccgaagcaucugcaagguccuccggucgguuucugccuguaacaguggcaacagcgcgaugcgagccucgaaguccucgauuuguaggcgccugcgcucacgguuccacuucauuaugcuccagugcccguagaucaggguuccaaucccuauggccagcaugcuguagcccgacaguccucgacgcggcaaguuccguu

SEQ ID NO: 13 - RNA, 500 nt, Homo sapiens
guguggcugccuucgagggucagguuggacaagcugcauacucugcuuccaaggggggaauagugggcaugacacugcccauugcucgggaucuggcucccauagguauccgggugaugaccauugccccaggucuguuuggcaccccacugcugaccagccucccagagaaagugugcaacuucuuggccagccaagugcccuucccuagccgacugggugacccugcugaguaugcucaccucguacaggccaucaucgagaacccauuccucaauggagaggucauccggcuggauggggccauucguaugcagccuugaagggagaaggcagagaaaacacacgcuccucugcccuuccuuucccugggguacuacucuccagcuugggaggaagcccaguagccauuuuguaacugccuaccagucgcccucugugccuaauaaagucucuuuuucucacaaaaaaaaaaaaaaaaaaaaaaaaaaaaaaaaaaa

SEQ ID NO: 14 - DNA, 500 bp, Homo sapiens
tttttttttttttttttttttttttttttttttttgtgagaaaaagagactttattaggcacagagggcgactggtaggcagttacaaaatggctactgggcttcctcccaagctggagagtagtaccccagggaaaggaagggcagaggagcgtgtgttttctctgccttctcccttcaaggctgcatacgaatggccccatccagccggatgacctctccattgaggaatgggttctcgatgatggcctgtacgaggtgagcatactcagcagggtcacccagtcggctagggaagggcacttggctggccaagaagttgcacactttctctgggaggctggtcagcagtggggtgccaaacagacctggggcaatggtcatcacccggatacctatgggagccagatcccgagcaatgggcagtgtcatgcccactattccccccttggaagcagagtatgcagcttgtccaacctgaccctcgaaggcagccacac

SEQ ID NO: 15 - DNA, 500 bp, Homo sapiens
gtgtggctgccttcgagggtcaggttggacaagctgcatactctgcttccaaggggggaatagtgggcatgacactgcccattgctcgggatctggctcccataggtatccgggtgatgaccattgccccaggtctgtttggcaccccactgctgaccagcctcccagagaaagtgtgcaacttcttggccagccaagtgcccttccctagccgactgggtgaccctgctgagtatgctcacctcgtacaggccatcatcgagaacccattcctcaatggagaggtcatccggctggatggggccattcgtatgcagccttgaagggagaaggcagagaaaacacacgctcctctgcccttcctttccctggggtactactctccagcttgggaggaagcccagtagccattttgtaactgcctaccagtcgccctctgtgcctaataaagtctctttttctcacaaaaaaaaaaaaaaaaaaaaaaaaaaaaaaaaaaa

SEQ ID NO: 16 - RNA, 500 nt, Homo sapiens
uuuuuuuuuuuuuuuuuuuuuuuuuuuuuuuuuuugugagaaaaagagacuuuauuaggcacagagggcgacugguaggcaguuacaaaauggcuacugggcuuccucccaagcuggagaguaguaccccagggaaaggaagggcagaggagcguguguuuucucugccuucucccuucaaggcugcauacgaauggccccauccagccggaugaccucuccauugaggaauggguucucgaugauggccuguacgaggugagcauacucagcagggucacccagucggcuagggaagggcacuuggcuggccaagaaguugcacacuuucucugggaggcuggucagcaguggggugccaaacagaccuggggcaauggucaucacccggauaccuaugggagccagaucccgagcaaugggcagugucaugcccacuauuccccccuuggaagcagaguaugcagcuuguccaaccugacccucgaaggcagccacac